(19) United States
(12) Patent Application Publication
(10) Pub. No.: US 2022/0125893 A1
(43) Pub. Date: Apr. 28, 2022

(54) METHODS FOR TREATING ARTHRITIS USING SPL-108 PEPTIDE

(71) Applicants: Splash Pharmaceuticals, Inc., San Diego, CA (US); The Regents of the University of California, Oakland, CA (US)

(72) Inventors: Malcolm Finlayson, San Diego, CA (US); David Nelson, San Diego, CA (US); Monica Guma, La Jolla, CA (US)

(21) Appl. No.: 17/526,776

(22) Filed: Nov. 15, 2021

Related U.S. Application Data
(63) Continuation-in-part of application No. PCT/US20/32673, filed on May 13, 2020.
(60) Provisional application No. 62/847,059, filed on May 13, 2019.

(51) Int. Cl. A61K 38/48; A61P 19/02; C12N 9/64

(52) U.S. Cl. CPC A61K 38/482; C12Y 304/21031; C12N 9/6408; A61P 19/02

(57) ABSTRACT

This invention relates to methods of treatment for arthritis using SPL-108 polypeptide. In particular, this invention relates to such methods of treatment by the administration of an SPL-108 polypeptide (also known as A6 peptide, a CD44 modulating peptide) or a variant thereof alone or in combination with a standard-of-care treatment for arthritis to alleviate and/or prevent symptoms of arthritis and/or to avoid side effects commonly associated with treating arthritis. Such methods of the present invention are used to treat inflammatory conditions such as rheumatoid arthritis, osteoarthritis, reactive arthritis, and/or psoriatic arthritis.

### CROSS-REFERENCES TO RELATED APPLICATIONS

This application is a continuation-in-part and claims benefit of PCT Application No. PCT/US20/32673, filed May 13, 2020, which claims benefit of U.S. Provisional Patent Application No. 62/847,059 filed May 13, 2019, the specification(s) of which is/are incorporated herein in their entirety by reference.

### STATEMENT REGARDING FEDERALLY SPONSORED RESEARCH OR DEVELOPMENT

This invention was made with government support under Grant No. R01 AR073324, awarded by National Institutes of Health. The government has certain rights in the invention.

### REFERENCE TO A SEQUENCE LISTING

Applicant asserts that the paper copy of the Sequence Listing is identical to the Sequence Listing in computer readable form found on the accompanying computer file, entitled SPLASH_19_06_PCT_CIP_Sequence_Listing_ST25. The content of the sequence listing is incorporated herein by reference in its entirety.

### BACKGROUND OF THE INVENTION

Arthritis is a family of diseases resulting in inflammation and pain at body joints and possibly other internal organs. The effects of arthritis can be debilitating to a subject's quality of life and effective treatments for many members of the arthritis family of diseases are limited and are of varying degrees of effectiveness. Bone loss, or alteration in bone turnover, can result from, or be associated with, many types of arthritis, including rheumatoid arthritis and osteoarthritis.

### FIELD OF THE INVENTION

This invention relates to methods of treatment for arthritis using SPL-108 polypeptide. In particular, this invention relates to such methods of treatment by the administration of an SPL-108 polypeptide (also known as A6 peptide, a CD44 modulating peptide; FIG. 1) or a variant thereof alone or in combination with a standard-of-care treatment for arthritis to alleviate and/or prevent symptoms of arthritis and/or to avoid side effects commonly associated with treating arthritis. Such methods of the present invention are used to treat inflammatory conditions such as rheumatoid arthritis, osteoarthritis, reactive arthritis, and/or psoriatic arthritis. In some embodiments, the standard-of-care treatments may comprise analgesics, anti-inflammatory agents, corticosteroids, disease modifying antirheumatic drugs (DMARDs), biological response modifiers, and/or physical therapy.

### BACKGROUND ART

Rheumatoid arthritis is a chronic, systemic and articular inflammatory disorder characterized by weakening of the joint capsules and ligaments, followed by destruction of cartilage, ligaments, tendon and bone, and a decrease in viscosity and other alterations in the synovial membrane and fluid. FIG. 2 shows schematic illustrations of a normal joint, a joint with osteoarthritis, and a joint with rheumatoid arthritis.

Rheumatoid arthritis symptoms include systemic weakness, fatigue, localized pain, and stiffness, weakness, Swelling, and deformation of the joints of the body. Rheumatoid arthritis is most common in women in the fourth to sixth decade of life. The pathogenesis of rheumatoid arthritis, leading to the destruction of the joints, is characterized by two phases: 1) an exudative phase involving the microcirculation of the synovial cells that allow an influx of plasma proteins and cellular elements into the joint and 2) a chronic inflammatory phase occurring in the sub-synovium and sub-chondral bone, characterized by pannus (granulation tissue) formation in the joint space, bone erosion, and cartilage destruction. The pannus contains proliferating fibroblast-like synoviocytes (FLSs) and macrophages that produce proinflammatory cytokines such as factor-alpha tumor necrosis (TNF-alpha) and interleukin-1 (IL-1). The pannus may form adhesions and scar tissue which causes the joint deformities characteristic of rheumatoid arthritis. Current rheumatoid arthritis treatment consists predominantly of symptomatic relief by administration of non-steroidal anti-inflammatory drugs (NSAIDs). NSAID treatment is mainly effective in the early stages of rheumatoid arthritis, it is unlikely it will produce suppression of joint inflammation if the disease is present for more than one year. Gold, methotrexate, immunosuppressants and corticosteroids also have been tried with limited success.

Osteoarthritis is a disorder of the movable joints characterized by deterioration and abrasion of articular cartilage, as well as by formation of new bone at the joint periphery. As osteoarthritis progresses, the surface of the articular cartilage is disrupted and wear-particles gain access to the synovial fluid which in turn stimulates phagocytosis by macrophage cells, inducing an inflammatory response in osteoarthritis. Common clinical symptoms of osteoarthritis include cartilaginous and bony enlargements of the finger joints and stiffness on awakening and painful movement.

Prior treatments for arthritis typically include standard-of-care treatments comprising analgesics, anti-inflammatory agents (e.g., NSAIDs), corticosteroids, disease modifying antirheumatic drugs (DMARDs), biological response modifiers, and/or physical therapy (e.g., exercise). The administration of NSAIDs is a common initial approach for relieving symptoms of arthritis. However, numerous side effects are associated with taking NSAIDs for prolonged periods, and/or in excessive doses. These common side effects include, but are not limited to, gastrointestinal disorders including gastrointestinal ulcerations, bleeding, and perforations, renal disease, hepatic disease; ocular disturbances, and physiological and psychological central nervous system effects. In addition, some oral NSAIDs may increase the risk of a heart attack or stroke.

Another common approach is the use of nonspecific cytotoxic immunosuppressive drugs. These drugs suppress the entire immune system and are incapable of selectively suppressing the abnormal autoimmune response. Non-limiting examples of such immunosuppressive drugs include methotrexate, cyclophosphamide, Imuran (azathioprine) and cyclosporin A. This global restraint of the immune system over time increases the risk of infection. Prolonged therapy with these nonspecific cytotoxic immunosuppressive drugs entails toxic side effects, including increased tendency towards development of certain malignancies, kidney failure, diabetes and liver function disorders. Moreover, cytotoxic immunosuppressive drug therapy merely slows down the progression of the disease, which resumes at an accelerated pace after the therapy is discontinued. For example, about six weeks after such a drug is discontinued, the patient deteriorates to the same stage as before the treatment was begun. In addition, the effectiveness of these drugs is self-limiting; they gradually cease being effective after about 2-5 years. Steroid compounds such as prednisone and methylprednisolone (which are also non-specific immunosuppressive and anti-inflammatory drugs) are also used for symptomatic relief. Steroids also have significant toxic side effects associated with their long-term use including increased appetite, weight gain, upset stomach, ulcers, and osteoporosis.

CD44 is a cell-surface glycoprotein involved in many vital normal bioactivities, including the interaction between cells and extracellular tissues, the support of cell migration in blood vessels and inside tissues, the presentation of growth factors, cytokines, chemokines and enzymes to other cells or to the surrounding tissues, and signal transmission from the cell surface to its interior, leading to apoptosis or cell survival and proliferation. As such, CD44 plays a role in a diverse range of physiological and pathological processes, including cell-cell and cell-extracellular matrix interactions, cell migration, lymphocyte homing, leukocyte activation, hemopoiesis, presentation of chemokines and growth factors, and metastatic spread. CD44 also is the main cell surface receptor for hyaluronate/hyaluronic acid (HA), as well as collagen and fibronectin.

Further, CD44 has been implicated to play a central role in arthritis and is expressed in fibroblasts (fibroblast synoviocytes; FLSs), including osteoarthritic and rheumatoid arthritic synoviocytes, and joint macrophages. In animal studies, CD44 overexpressing mice showed increased arthritis and antibodies to CD44 improved the arthritic condition. Other animal studies showed that SPL-108 inhibited arthritis-induced osteoclastogenesis.

Current treatments for arthritis are non-specific, of limited efficacy, involve significant toxic side effects, and cannot be used indefinitely. Accordingly, the present invention features novel treatments and novel therapeutic compositions for human autoimmune arthritis that do not suffer from one or more of the drawbacks identified above.

### BRIEF SUMMARY OF THE INVENTION

It is an objective of the present invention to provide methods of using SPL-108 (or A6) polypeptide, a variant thereof, or a CD44-modulating peptides alone or in combination with other agents that allow for effectively treating arthritis, alleviating symptoms of arthritis, and avoiding side effects or allergic reactions associated with treating arthritis, as specified in the independent claims. Embodiments of the invention are given in the dependent claims. Embodiments of the present invention can be freely combined with each other if they are not mutually exclusive.

One of the unique and inventive technical features of the present invention is the use of a targeted peptide (e.g., for CD44 expressing cells) in the treatment of arthritis (particular features, sequence of features, or combination of features). Without wishing to limit the invention to any theory or mechanism, it is believed that the technical feature of the present invention advantageously provides for alleviating symptoms of arthritis while avoiding common side effects associated with treating arthritis. None of the presently known prior references or work has the unique inventive technical feature of the present invention.

In the existing literature, CD44 is not generally regarded as a selective target, due to its broad interaction with numerous cellular functions. The development of CD44 targeting therapeutics has not been successful in the past, in part due to the unwanted collateral effects on biologically important CD44-related signaling pathways, as demonstrated by various abandoned anti-CD44 clinical programs. In contrast to previously-reported CD44 targeting strategies that have proven to be ineffective and/or excessively toxic, the present invention features a method of targeting CD44 without the untoward side effects. The present invention features the use of SPL-108, which, without being bound by theory or mechanism, is currently believed to target a unique region on the CD44 receptor and to inhibit the cell invasion/migration component of arthritis. SPL-108 is currently believed, again without being bound by theory or mechanism, to inhibit the second proinflammatory cytokine component of arthritis. Thus, SPL-108 surprisingly produces a more global effect, with only very minimal or no adverse toxicity or side effects. Other advantages may become apparent to one skilled in the art upon practice of the embodiments described and enabled herein.

The present invention features a method of treating a subject afflicted with arthritis, a method of alleviating symptoms of arthritis, and a method of avoiding side effects or allergic reactions associated with treating arthritis. In preferred embodiments, these methods comprise, during a first period, administering a polypeptide, which polypeptide is Ac-KPSSPPEE-NH₂ (SEQ ID NO:1), an active variant thereof, or a CD44-modulating peptide, to the subject afflicted with arthritis at an effective daily dose over a period of time sufficient to effectively treat arthritis. In some embodiments, the polypeptide is Ac-KPSSPPEE-NH₂ (SEQ ID NO:1), and the effective dose is from about 10 mg to about 300 mg per day. In some embodiments, the first period comprises a period of time sufficient to alleviate one or more clinical symptoms of arthritis comprising at least 1 day, at least 2 days, at least 7 days, at least 14 days, at least 42 days, at least 60 days, or until the one or more clinical symptoms of arthritis are no longer alleviated in the subject. In some embodiments, the arthritis comprises osteoarthritis, reactive arthritis, rheumatoid arthritis, psoriatic arthritis or combination thereof. In some embodiments, the administration of SEQ ID NO:1 is parenteral administration comprising subcutaneous administration, intradermal administration, and/or intranasal administration. In some embodiments, the dose of SEQ ID NO:1 ranges from 5 mg to 50 mg, administered twice daily. In some embodiments, the dose of SEQ ID NO:1 ranges from 50 mg to 100 mg, administered twice daily. In some embodiments, the dose of SEQ ID NO:1 ranges from 100 mg to 150 mg, administered twice daily. In some embodiments, the method further comprises, during a second period, giving the subject a drug holiday from administering SEQ ID NO:1. In some embodiments, the drug holiday comprises withholding SEQ ID NO:1 administration for a period of time. In some embodiments, the drug holiday comprises withholding SEQ ID NO:1 administration for a period of time, wherein one or more clinical symptoms of arthritis continue to be alleviated. In some embodiments, the method further comprises, during a second period, reducing the daily dose of SEQ ID NO:1. In some embodiments, the method further comprises, during the second period, reducing the daily dose of SEQ ID NO:1 to from 10 mg to 5 mg per day. In some embodiments, the method further comprises, during a second period, increasing the daily dose of SEQ ID NO:1. In some embodiments, the method further comprises during the second period, increasing the daily dose of SEQ ID NO:1 to from 300 mg to 600 mg per day. In some embodiments, the second period comprises at least 1 day, at least 2 days, at least 7 days, at least 14 days, at least 42 days, at least 60 days, or until one or more clinical symptoms of arthritis are no longer alleviated in the subject. In some embodiments, effectively treating arthritis in a subject comprises alleviating clinical symptoms of arthritis comprising one or more of joint pain, joint stiffness, joint swelling or inflammation, joint tenderness, joint redness, or a limited range of motion. In some embodiments, alleviating the one or more clinical symptoms of arthritis comprises alleviating one or more of reducing joint pain, reducing joint stiffness, reducing joint swelling, reducing joint redness, or increasing range of motion. In some embodiments, the joint pain is reduced by at least about 20%. In some embodiments, the joint stiffness is reduced by at least about 20%. In some embodiments, the joint swelling is reduced by at least about 20%. In some embodiments, the joint redness is reduced by at least about 20%. In some embodiments, the range of motion is increased by at least about 20%. In some embodiments, the polypeptide comprises a variant sequence of SEQ ID NO:1, wherein the variant sequence comprises one or more amino acid mutation with respect to SEQ ID NO:1 selected from: (a) K¹ to A; (b) P², P⁵, P⁶, or a combination thereof to A; (c) S³, S⁴, or S³ and S⁴ to A; or (d) E⁷, E³, or E⁷ and E⁸ to A, wherein the mutation retains CD44 modulating activity substantially similar to or greater than the polypeptide of SEQ ID NO:1. In some embodiments, mutation retains CD44 modulating activity of about equal to or greater than the polypeptide of SEQ ID NO:1.

The present invention provides a method of treating arthritis or alleviating one or more clinical symptoms of arthritis in a subject in need thereof, said method comprising, during a first period, administering a polypeptide, wherein said polypeptide is Ac-KPSSPPEE-NH2 (SEQ ID NO:1), an active variant thereof, or a CD44-modulating peptide, to said subject at an effective daily dose over a period of time sufficient to effectively treat arthritis or alleviate one or more clinical symptoms of arthritis. In some embodiments, the polypeptide is Ac-KPSSPPEE-NH2 (SEQ ID NO:1), and said effective dose is from about 10 mg to about 300 mg per day. In some embodiments, the polypeptide comprises a variant sequence of SEQ ID NO:1, wherein the variant sequence comprises one or more amino acid mutation with respect to SEQ ID NO:1 selected from: (a) K1 to A; (b) P2, P5, P6, or a combination thereof to A; (c) S3, S4, or S3 and S4 to A; or (d) E7, E8, or E7 and E8 to A, wherein said mutation retains CD44-modulating activity substantially similar to or greater than said polypeptide of SEQ ID NO:1.

The present invention also provides a method of treating arthritis or alleviating one or more clinical symptoms of arthritis in a subject in need thereof, said method comprising, during a first period, administering a polypeptide, wherein said polypeptide is Ac-KPSSPPEE-NH2 (SEQ ID NO:1), an active variant thereof, or a CD44-modulating peptide, to said subject at a daily dose from 10 mg to 300 mg per day; and administering an antiproliferative agent to said subject at a weekly dose of from 10 mg/m2 to 250 mg/m2 per week, wherein the polypeptide and antiproliferative agent administered during a first period are effective for treating arthritis or alleviating one or more clinical symptoms of arthritis.

In some embodiments, the first period comprises a period of time sufficient to alleviate one or more clinical symptoms of arthritis comprising at least 1 day, at least 2 days, at least 7 days, at least 14 days, at least 42 days, at least 60 days, or until said one or more clinical symptoms of arthritis are no longer alleviated in said subject. In some embodiments, wherein said arthritis comprises osteoarthritis, reactive arthritis, rheumatoid arthritis, psoriatic arthritis or combination thereof. In some embodiments, administration of SEQ ID NO:1 is parenteral administration. In some embodiments, the method further comprises, during a second period, giving said subject a drug holiday from administering SEQ ID NO:1. In some embodiments, the drug holiday comprises withholding SEQ ID NO:1 administration for a period of time. In some embodiments, the method further comprises, during a second period, reducing said daily dose of SEQ ID NO:1. In some embodiments, the method further comprises, during a second period, increasing said daily dose of SEQ ID NO:1. In some embodiments, the method further comprises, during said second period, increasing the daily dose of SEQ ID NO:1 to from 300 mg to 600 mg per day.

In some embodiments, the one or more clinical symptoms of arthritis is selected from: joint pain, joint stiffness, joint swelling, joint redness, reduced range of motion. In some embodiments, joint pain is reduced by at least about 20%. In some embodiments, joint stiffness is reduced by at least about 20%. In some embodiments, joint redness is reduced by at least about 20%. In some embodiments, range of motion is increased by at least about 20%.

In some embodiments, the polypeptide comprises a variant sequence of SEQ ID NO:1, wherein the variant sequence comprises one or more amino acid mutation with respect to SEQ ID NO:1 selected from: (a) K1 to A; (b) P2, P5, P6, or a combination thereof to A; (c) S3, S4, or S3 and S4 to A; or (d) E7, E8, or E7 and E8 to A, wherein said mutation retains CD44-modulating activity substantially similar to or greater than said polypeptide of SEQ ID NO:1.

In some embodiments, the antiproliferative agent is an anti-microtubule agent. In some embodiments, the antimicrotubule agent is a taxane.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 illustrates that the polypeptide of SEQ ID NO:1 shares sequence homology with a portion of the Link-Domain of CD44 (CD44 amino acid residues 120-NASAPPEE-127 (SEQ ID NO: 8).

FIG. 2 shows schematic illustrations of a normal joint, a joint with osteoarthritis, and a joint with rheumatoid arthritis.

FIG. 3 shows a mouse model of Collagen-induced Arthritis (CIA).

FIG. 4 shows non-limiting examples of a non-diseased paw and a CIA-diseased paw with associated clinical scores.

FIG. 5 shows the effect of SPL-108 (A6) on the body weight (in grams) of CIA mice at disease onset through 11 days post-disease onset.

FIG. 6 shows the effect of SPL-108 (A6) on paw swelling (in mm) of mice at disease onset through 11 days post-disease onset.

FIG. 7 shows the effect of SPL-108 (A6; 50 mg and 100 mg) on clinical scores of CIA mice at disease onset through 11 days post-disease onset.

FIGS. 8A and 8B show the effect of SPL-108 (A6; 0.1, 1.0, 10.0, 100 μM) on Matrigel invasion of fibroblast-like synoviocytes (FLSs).

FIG. 9 shows the effect of SPL-108 (A6; 0.1, 1.0, 10.0, 100 μM) on Scratch-Wound migration of fibroblast-like synoviocytes (FLSs).

FIGS. 10A, 10B, 10C, 10D, and 10E show histology and histological scores in ankles collected at the end of the experiments. At the end of the experiment, ankle bones were collected and decalcified in EDTA for 10 days. Decalcified bones were embedded and sagittal sections obtained and stained. H&E staining was used to help score cell infiltration, synovial thickness, and bone damage (FIG. 10A). In the figure, safranin staining will help to score cartilage damage. Black arrows show infiltration in arthritic mice treated with PBS compared to control mice without arthritis. White arrows show lack of safranin staining in the arthritic mice cartilage because of proteoglycan degradation compared to control mice without arthritis.

### DETAILED DESCRIPTION OF THE INVENTION

All patents, applications, published applications and other publications are incorporated by reference in their entirety and for all purposes. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

The terms “polypeptide” and “protein” are used interchangeably herein and refer to any molecule that includes at least 2 or more amino acids.

As used herein, “administering” and the like refer to the act physically delivering a composition or other therapy (e.g. an arthritis therapy, NSAIDs) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

The term “coadministration” refers to administration of two or more agents (e.g., a polypeptide described herein and another active agent such as an agent e.g., (NSAID, DMARD) or other therapy (e.g. physical therapy) described herein). The timing of coadministration depends in part on the combination and compositions or other therapies administered and can include administration at the same time, just prior to, or just after the administration of one or more additional therapies, for example therapies such as analgesics, anti-inflammatory agents, corticosteroids, DMARDs, biological response modifiers, and physical therapy. Coadministration is meant to include simultaneous or sequential administration of a composition or therapy individually or in combination (more than one polypeptide described herein or an agent for treating arthritis described herein or physical therapy as described herein). Coadministration can include administration of two or more agents where the agents are optionally combined with other active substances (e.g., to reduce metabolic degradation). The polypeptides, agents for treating arthritis and physical therapies described herein can be used in combination with one another, with other active agents known to be useful in treating a disease associated with cells expressing a particular kinase as described herein, or with adjunctive agents that cannot be effective alone, but can contribute to the efficacy of the active agent.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “arthritis” refers to any physiological condition in mammals characterized by inflammation of one or more joints. The main symptoms of arthritis are joint pain, joint stiffness, joint swelling, joint redness, decreased range of motion, which typically worsen with age. Arthritis types described herein include the most common types of arthritis, osteoarthritis and rheumatoid arthritis. Other types of arthritis described herein include psoriatic arthritis, reactive arthritis, bursitis, Juvenile arthritis, Ankylosing spondylitis, Lyme disease, and Lupus.

An improvement in the arthritis or arthritis-related disease or an alleviation of clinical symptoms can be characterized as a complete alleviation of one or more of symptoms, where the subject has or displays no clinical symptoms of arthritis, or a partial alleviation of symptoms, where the subject displays at least a 20% improvement in one or more of clinical symptoms of arthritis.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “preventing” refers to the treatment with or administration of a polypeptide or agent (e.g. anti-proliferative agent described herein) provided herein, with or without other additional active agent (e.g. a standard-of-care agent for arthritis), prior to the onset of symptoms, particularly to patients at risk of arthritis and/or other disorders described herein. The term also refers to coadministration of a polypeptide with other therapies including standard-of-care therapies for arthritis as described herein. It should be understood that the polypeptides described herein can be coadministered with one or more standard-of-care therapies for arthritis described herein. The term prevention includes the inhibition or reduction of a symptom of the particular disease, as well as a reduced incidence of a symptom of the particular disease (e.g. by comparison to historical data for a given subject, or population data for similar subjects). Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

A prophylactically effective amount of a polypeptide or agent (e.g. an anti-proliferative agent described herein) means an amount of therapeutic agent, alone or in combination with other agents, which provides a prophylactic benefit in the inhibition or reduced incidence of a symptom of a disease or recurrence of a disease. The term also refers to coadministration of a polypeptide described herein with other therapies including standard-of-care therapies for arthritis as described herein. The term prophylactically effective amount can encompass an amount that improves overall prophylaxis or enhances the prophylactic efficacy of another prophylactic agent.

The term “effective amount” as used herein refers to the amount of a therapy (e.g., a composition, SPL-108, and/or or standard-of-care therapies for arthritis therapy provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of a polypeptide described herein, or a standard-of-care treatment described herein, is an amount sufficient to provide a therapeutic benefit in the treatment or management of an arthritis, or to delay or minimize one or more symptoms associated with the presence of the arthritis. A therapeutically effective amount of a polypeptide described herein or a standard-of-care therapy for arthritis described herein means an amount of therapeutic agent, alone or in combination with other therapies, which provides a therapeutic benefit in the treatment or management of the arthritis. The term “therapeutically effective amount” can encompass an amount that improves overall therapy, reduces or avoids symptoms or causes of arthritis, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, physical therapy, and/or other therapies useful in the prevention, management, treatment and/or amelioration of a given disease, disorder or condition known to one of skill in the art such as medical personnel.

A regimen is a protocol for dosing and timing the administration of one or more therapies (e.g., combinations described herein, another active agent such as for example an anti-proliferative agent described herein, or a standard-of-care therapy for arthritis described herein) for treating a disease, disorder, or condition described herein. A regimen can include periods of active administration and periods of rest as known in the art. Active administration periods include administration of combinations and compositions described herein and the duration of time of efficacy of such combinations, compositions, and physical therapies. Rest periods of regimens described herein include a period of time in which no agent (e.g., a polypeptide described herein or a standard-of-care agent for arthritis agent described herein) is actively administered, and in certain instances, includes time periods where the efficacy of such agents can be minimal. Rest periods of regimens described herein can include a period of time in which no physical therapy is actively performed. Combination of active administration and rest in regimens described herein can increase the efficacy and/or duration of administration of the combinations and compositions described herein.

The term “pharmaceutically acceptable” as used herein refers to physiologically acceptable compounds, agents, or ingredients recognized by a regulatory agency of the Federal or state government, or another governmental agency with authorization for such approval, or and an agent listed in the U.S. Pharmacopeia, European Pharmacopeia or other generally recognized Pharmacopeia for use in animals, and more particularly in humans.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, sucrose, glucose, binders, fillers, disintegrants, lubricants, coatings, sweeteners, flavors, alcohols, oils, gelatins, carbohydrates such as amylose or starch, fatty acid esters, hydroxymethylcellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6th Ed., The Pharmaceutical Press, RPS Publishing (2009). The terms binder, filler, disintegrant, and lubricant are used in accordance with the plain and ordinary meaning within the art.

In certain embodiments, a pharmaceutically acceptable excipient may be incompatible (e.g., cross-reacts) with other excipients or active agents described herein. In some embodiments, magnesium stearate, croscarmellose sodium, lactose, excipients comprising Mg, Ca, K, Li, or Nucleic acid, acesulfame potassium, ammonium alginate, calcium acetate, calcium alginate, calcium carbonate, calcium chloride, calcium lactate, calcium phosphate, calcium silicate, calcium stearate, calcium sulfate, carboxymethylcellulose calcium, carboxymethylcellulose sodium, docusate sodium, glycine, kaolin, magnesium aluminum silicate, magnesium carbonate, magnesium oxide, magnesium silicate, magnesium trisilicate, polacrilin potassium, polymethacrylates, potassium alginate, potassium benzoate, potassium bicarbonate, potassium chloride, potassium citrate, sodium alginate, sodium benzoate, sodium chloride, sodium lauryl sulfate, sodium starch glycolate, sodium stearyl fumarate, sulfobutylether beta-cyclodextrin, sodium stearate, talc, or zinc stearate are incompatible in the dosage forms described herein.

As used herein, As used herein, the term “A6” or “A6 polypeptide” or “SPL-108” is intended to mean a polypeptide having substantially the amino acid sequence Lys-Pro-Ser-Ser-Pro-Pro-Glu-Glu (also abbreviated in single letter amino acid code as KPSSPPEE, as in SEQ ID NO:7) or a substitution variant, addition variant, or chemical derivative thereof including peptidomimetics. An A6 polypeptide is the subject matter of U.S. Pat. Nos. 5,994,309; 6,696,416; and 6,963,587. An A6 polypeptide of the invention exhibits one or more of the following activities: (a) at least about 20% of the biological activity of SEQ ID NO:1 or a capped variant as described below in one or more of the following in vitro bioassays: (i) invasion in a Matrigel R. assay; (ii) endothelial tube formation on Matrigel®, or (iii) endothelial tube formation on a fibrin matrix in the presence of basic fibroblast growth factor and vascular endothelial growth factor, or (b) binding activity such that it competes with labeled SEQ ID NO:1 or a capped variant for binding to a cell or molecule which has a binding site for SEQ ID NO:1.

A capped variant of an A6 polypeptide of the invention refers to A6 having chemical moieties at either or both of its amino or carboxyl termini. The moieties can include, for example, chemical groups such as acetyl (Ac) and amido (Am) groups. A particularly useful capped A6 polypeptide includes an acetyl group bound to the nitrogen at the amino-terminus and an amido group bound to the C-terminal carboxyl group. This capped polypeptide can be written as Ac-KPSSPPEE-Am (SEQ ID NO:1). In specific embodiments, the invention also provides homologous A6 polypeptide of SEQ ID NO:7.

As used herein, a “CD44-modulating polypeptide” refers to a polypeptide that binds to CD44 and modulates its activity (e.g., signaling activity). A CD44-modulating polypeptide can be a polypeptide sequence described herein or, in some embodiments, an antibody that specifically binds to CD44 and inhibits its downstream signaling activity. In one embodiment, a CD44-modulating polypeptide can be a polypeptide sequence described herein or, in some embodiments, an antibody that disrupts or inhibits signaling activity of a CD44 dependent co-receptor. In certain instances, the CD44 dependent co-receptor is a receptor tyrosine kinase (RTK) such as, for example, Met, Ran, or VEGFR. In still another embodiment a CD44-modulating polypeptide can be a polypeptide sequence described herein or, in some embodiments, an antibody that disrupts CD44 co-receptor function or association of a CD44 co-receptor with CD44 or another signaling protein. In one embodiment, a CD44-modulating polypeptide described herein binds to CD44 and inhibits CD44 signaling activity or association with one or more ABC transporters. The ABC transporter can be a multidrug resistant protein (e.g., MDR1). In certain embodiments, CD44 levels can be elevated upon radiation therapy.

Exemplary CD44-modulating polypeptides include polypeptides having homology to the CD44-v6 region of human CD44. Such peptides can include substitution variants, addition variants, or chemical derivatives thereof including peptidomimetics. In one embodiment, the CD44-modulating polypeptide described herein is a polypeptide having the amino acid sequence of Ac-KPSSPPEE-NH2 (SEQ ID NO:1), Ac-NASAPPEE-NH2 (SEQ ID NO:2), QETWFQNGWQGKNP (SEQ ID NO:3), KEKWFENEWQGKNP (SEQ ID NO:4), or KEQWFGNRWHEGYR (SEQ ID NO:5), KPSSPPEE (SEQ ID NO:7), NASAPPEE (SEQ ID NO: 8). Another CD44-modulating polypeptide can be QIRQQPRDPPTETLELEVSPDPAS (SEQ ID NO:6). Such exemplary peptides can include substitution variants, addition variants, or chemical derivatives thereof including peptidomimetics. Other exemplary CD44-modulating peptides include those set forth in U.S. Pat. Nos. 5,994,309; 6,696,416; 6,963,587; 8,313,914; 8,697,629, each of which is incorporated herein in its entirety.

The term “peptidomimetic,” as used herein, means a peptide-like molecule that has the activity of the polypeptide upon which it is structurally based. Such peptidomimetics include chemically modified peptides, peptide-like molecules containing non-naturally occurring amino acids, and peptoids, and have an activity such as the selective homing activity of the polypeptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons (1995), pages 803-861).

The term “prodrug” refers to a compound or polypeptide that is made more active in vivo through metabolism of a precursor drug. CD44-modulating polypeptides described herein can exist as prodrugs, as described in, for example, Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the polypeptides described herein are structurally modified forms of the polypeptide that readily undergo chemical changes under physiological conditions to provide the active polypeptide. Additionally, prodrugs can be converted to the active polypeptide by chemical or biochemical methods in an ex vivo environment.

The medications used to treat arthritis vary depending on the type of arthritis. In preferred embodiments, commonly used arthritis medications (or standard-of-care) include analgesics, anti-inflammatory drugs, counter-irritants, DMARDS, and biologic response modifiers.

Analgesics. These medications help reduce pain, but have no effect on inflammation. Examples include acetaminophen (Tylenol, others), tramadol (Ultram, Ultracet, others) and narcotics containing oxycodone (Percocet, Oxycontin, others) or hydrocodone (Norco, Vicoprofen, others).

Nonsteroidal anti-inflammatory drugs (NSAIDs). NSAIDs reduce both pain and inflammation. Over-the-counter NSAIDs include ibuprofen (Advil, Motrin IB, others) and naproxen sodium (Aleve). Some types of NSAIDs are available only by prescription. Oral NSAIDs can cause stomach irritation, and some may increase the risk of a heart attack or stroke. Some NSAIDs are also available as creams or gels, which can be rubbed on joints.

The non-steroidal anti-inflammatory agents suitable for use in the methods of treatment for arthritis described herein include all NSAIDs used to treat undesirable inflammation of body tissues. Suitable NSAIDs for use in the treatment regimens described herein include, but are not limited to, indole-based anti-inflammatory agents (including among others, indomethacin, indoxole and the like); salicylate-based anti-inflammatory agents (including among others, aspirin and the like); phenylacetic acid-based anti-inflammatory drugs (including, among others, fenoprofen, ketoprofen, MK-830 and the like), pyrazolidine-based anti-inflammatory agents (including, among others, phenylbutazone, oxyphenbutazone, and the like); and p-(isobutylphenyl)ace tic acid-based anti-inflammatory agents (including, among others, buprofen, ibufenac, and the like).

NSAIDS preferred for use in the methods of treatment described herein include, but are not limited to, salicylates, indomethacin, flurbiprofen, diclofenac, naproxen, piroxicam, tebufelone, and ibuprofen. Other NSAIDs suitable for use herein include, but are not limited to, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, cloniXin, ditrazol, epirizole fenoprofen, floctafenini, flufenamic acid, glaphenine, indoprofen, ketoprofen, meclofenamic acid, mefenamic acid, niflumic acid, phenacetin, salidifamides, sulindac, suprofen, and tolmetin. In addition to the particular NSAIDs described above, suitable NSAIDS also include any non-steroidal compound used to treat undesirable inflammation of body tissues.

Inflammation or the “inflammatory response” is the result of complex interconnected physiological events, including increased vascular permeability, fluid accumulation, and the migration of a changing population of inflammatory cells into the inflamed area. The clinical manifestations of inflammation include swelling (edema), increased local temperature, erythema, and pain. The inflammatory response can be triggered by any of a number of causative factors, including certain bacteria, radiation, hypersensitivity to chemical agents, arthritis, and arthritic-like conditions.

The inflammatory response is generally believed to be a primary defense mechanism in the body, but unchecked, can become excessive and can result in functional impairment. It is believed that most of the bone and joint destruction that occurs in arthritic conditions occurs during periods of flare, when inflammation is especially severe. As stated above, there are numerous side effects associated with taking NSAIDs for prolonged periods, and/or in excessive doses. These common side effects include, but are not limited to, gastrointestinal disorders including gastrointestinal ulcerations, bleeding, and perforations, renal disease, hepatic disease; ocular disturbances, and physiological and psychological central nervous system effects.

Counterirritants. Some varieties of creams and ointments contain menthol or capsaicin, the ingredient that makes hot peppers spicy. Rubbing these preparations on the skin over aching joint may interfere with the transmission of pain signals from the joint itself.

Disease-modifying antirheumatic drugs (DMARDs). Often used to treat rheumatoid arthritis, DMARDs slow or stop your immune system from attacking your joints. Examples include methotrexate (Trexall) and hydroxychloroquine (Plaquenil).

Biologic response modifiers. Typically used in conjunction with DMARDs, biologic response modifiers are genetically engineered drugs that target various protein molecules that are involved in the immune response. Examples include etanercept (Enbrel) and infliximab (Remicade).

Corticosteroids. This class of drug, which includes prednisone and cortisone, reduces inflammation and suppresses the immune system. Corticosteroids can be taken orally or be injected directly into the painful joint.

In other embodiments, the standard-of-care treatment comprises physical therapy. Exercises can improve range of motion and strengthen the muscles surrounding joints. In some cases, splints or braces may be warranted.

In some embodiments where conservative measures (e.g., medication, physical therapy) don't help, surgery may be an alternative treatment, such as joint repair, joint replacement, or joint fusion. For example, joint surfaces can be smoothed or realigned to reduce pain and improve function, for which procedures can often be performed arthroscopically, through small incisions over the joint. A joint replacement procedure removes damaged joint and replaces it with an artificial one. Joints most commonly replaced are hips and knees. A joint fusion procedure is more often used for smaller joints, such as those in the wrist, ankle and fingers. It removes the ends of the two bones in the joint and then locks those ends together until they heal into one rigid unit.

In other embodiments, the standard-of-care treatment comprises lifestyle and home remedies comprising weight loss, exercise, heat and cold, and assistive devices. For example, if the subject is obese, losing weight will reduce the stress on weight-bearing joints, which may increase mobility and limit future joint injury. In some embodiments, regular exercise can help keep joints flexible or heating pads or ice packs may help relieve arthritis pain. Using canes, walkers, raised toilet seats and other assistive devices can help protect joints and improve ability to perform daily tasks.

In some embodiments, the standard-of-care treatment for arthritis comprises alternative remedies such as acupuncture, glucosamine, yoga/tai chi, or massage. For example, acupuncture therapy uses fine needles inserted at specific points on the skin to reduce many types of pain, including that caused by some types of arthritis. Other embodiments may include administration of glucosamine, which relieves arthritis pain better than taking nothing, particularly in people who have moderate to severe pain. The slow, stretching movements associated with yoga and tai chi may help improve joint flexibility and range of motion in people with some types of arthritis. Massaging including light stroking and kneading of muscles may increase blood flow and warm affected joints, temporarily relieving pain.

The compositions for use in the methods and compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with antibodies. The preferred mode of administration is parenteral (e.g., intra-venous, subcutaneous, intraperitoneal, intramuscular). In a preferred embodiment, SPL-108 is administered by intravenous infusion or injection. In other preferred embodiments, SPL-108 is administered by intramuscular or subcutaneous injection or intranasally.

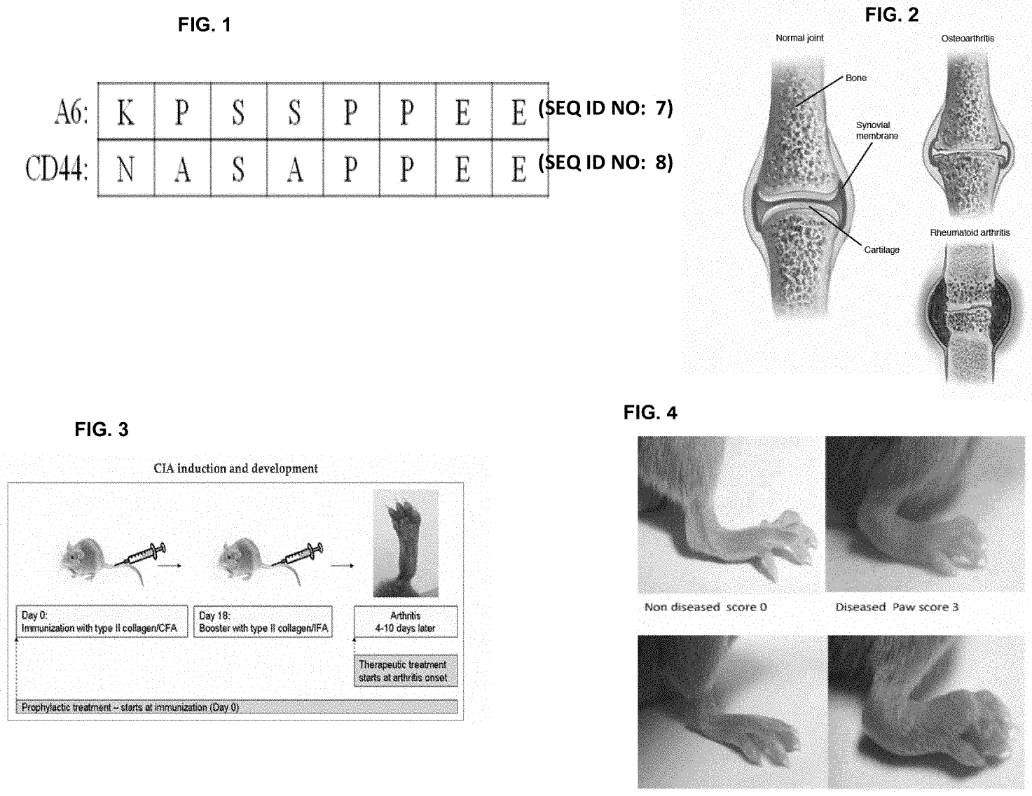

Referring now to FIGS. 1-9, the present invention features methods for treating arthritis, alleviating the symptoms of arthritis, and avoiding side effects commonly associated with treating arthritis.

Provided herein are methods of treating, preventing, managing, or alleviating arthritis or symptoms of an arthritis in a patient in need thereof. The methods may include single agent therapy (monotherapy) approach or a combination therapy. In particular embodiments, the method is for treating preventing, managing, or alleviating osteoarthritis or symptoms of osteoarthritis.

The present invention features a method for treating a subject afflicted with arthritis using a single agent (e.g., monotherapy). The method comprises, during a first period, administering a polypeptide of SEQ ID NO:1 (Ac-KPSSPPEE-NH₂) or a variant thereof, to the subject afflicted with arthritis at a daily dose of from about 10 mg to about 300 mg per day or administering a CD44-modulating peptide over a period of time sufficient to effectively treat arthritis.

The present invention also features a method for avoiding one or more side effects commonly associated with treating arthritis using a single agent (e.g., monotherapy). The method comprises, during a first period, administering a polypeptide of SEQ ID NO:1 or variant thereof, to a subject suffering from arthritis at a daily dose of from about 10 mg to about 300 mg per day or administering a CD44-modulating peptide over a period of time sufficient to avoid the one or more side effects commonly associated with treating arthritis.

The present invention further features a method for preventing arthritis in a subject at risk for developing arthritis using a single agent (e.g., monotherapy). The method comprises, during a first period, administering a polypeptide, of SEQ ID NO:1 or variant thereof, to the subject at risk for developing arthritis at a daily dose of from about 10 mg to about 300 mg per day or administering a CD44-modulating peptide over a period of time sufficient to prevent the development of one or more clinical symptoms of arthritis.

In specific embodiments, the present invention also features a method for alleviating one or more clinical symptoms of osteoarthritis using a single agent (e.g., monotherapy). The method comprises, during a first period, administering a polypeptide of SEQ ID NO:1 or a variant thereof, to a subject suffering from osteoarthritis at a daily dose of from about 10 mg to about 300 mg per day or administering a CD44-modulating peptide over a period of time sufficient to alleviate the one or more clinical symptoms of osteoarthritis.

In other embodiments, the present invention further features a method for treating a subject afflicted with arthritis using a combination of a polypeptide of SEQ ID NO:1 and a standard-of-care treatment (e.g., combination therapy). The method comprises, during a first period, administering a polypeptide of SEQ ID NO:1 or a variant thereof, to the subject at a daily dose of from about 10 mg to about 300 mg per day or a CD44-modulating peptide and administering a standard-of-care treatment for arthritis. The administration of SEQ ID NO:1 and standard-of-care is over a period of time sufficient to effectively treat the arthritis.

The present invention also features a method of alleviating one or more of clinical symptoms of arthritis in a subject afflicted with arthritis using a combination of a polypeptide of SEQ ID NO:1 and a standard-of-care treatment (e.g., combination therapy). The method comprises, during a first period, administering a polypeptide of SEQ ID NO:1, to the subject at a daily dose of from about 10 mg to about 300 mg per day or a CD44-modulating peptide and administering a standard-of-care treatment for arthritis. The administration of SEQ ID NO:1 and standard-of-care treatment is over a period of time sufficient to alleviate one or more clinical symptoms of arthritis.

The present invention also features a method for avoiding one or more side effects commonly associated with treating arthritis using combination therapy. The method comprises, during a first period, administering a polypeptide of SEQ ID NO:1 or variant thereof, to a subject suffering from arthritis at a daily dose of from about 10 mg to about 300 mg per day or a CD44-modulating peptide and administering a standard-of-care treatment for arthritis. The administration of SEQ ID NO:1 and standard-of-care is over a period of time sufficient to avoid the one or more side effects commonly associated with treating arthritis.

In some embodiments, the present invention features a method of preventing arthritis in a subject at risk for developing arthritis using combination therapy. The method comprises, during a first period, administering a polypeptide of SEQ ID NO:1, or variant thereof, to the subject at risk for developing arthritis at a daily dose of from about 10 mg to about 300 mg per day or a CD44-modulating peptide and administering a standard-of-care treatment for arthritis. The administration of SEQ ID NO:1 and standard-of-care treatment is over a period of time sufficient to prevent one or more clinical symptoms of arthritis.

In specific embodiments, the present invention also features a method of alleviating one or more clinical symptoms of osteoarthritis using combination therapy. The method comprises, during a first period, administering a polypeptide of SEQ ID NO:1 or variant thereof, to a subject suffering from osteoarthritis at a daily dose of from about 10 mg to about 300 mg per day of a CD44-modulating peptide and administering an antiproliferative agent to the subject at a weekly dose of from about 10 mg/m² to about 250 mg/m² per week.

In some embodiments an effective amount of a CD44-modulating polypeptide, or capped variant, is used in the treatment, prevention, or amelioration of arthritis. An effective amount may be from about 1 mg/kg to about 1000 mg/kg, or from about 5 mg/kg to about 500 mg/kg, between about 10 mg/kg to about 250 mg/kg. An effective amount can be determined by a physician upon consideration of such variables as age, weight, sex, and previous medical history of the subject. One skilled in the art will recognize that an amount between 1 mg/kg to about 1000 mg/kg can be administered including, without limitation, about 1, 2, 3, 4, 5, 6, 7, 8, 9, 10, 15, 20, 25, 30, 35, 40, 45, 50, 60, 70, 80, 90, 100, 120, 140, 150, 200, 250, 300, 350, 400, 450, 500, 550, 600, 650, 700, 750, 800, 850, 900, 950, and 1,000 mg/kg and any amount in between including fractions of a 1 mg/kg.

In some embodiments, the first period comprises a period of time sufficient to effectively treat the arthritis, alleviate one or more clinical symptoms of arthritis, and/or to avoid side effects commonly associated with treating arthritis. Some arthritis types include but are not limited to osteoarthritis, rheumatoid arthritis, reactive arthritis, psoriatic arthritis, or a combination thereof. The first period of time may comprise at least 1 day, at least 2 days, at least 7 days, at least 14 days, at least 42 days, at least 60 days, or until the one or more clinical symptoms of arthritis are no longer alleviated and/or until one or more of the side effects are no longer avoided in the subject.

In preferred embodiments. the administration of SEQ ID NO:1 is parenteral administration comprising subcutaneous administration, intradermal administration, and/or intranasal administration. In some embodiments, the dose of SEQ ID NO:1 ranges from 5 mg to 50 mg, administered twice daily, ranges from 50 mg to 100 mg, administered twice daily, or ranges from 100 mg to 150 mg, administered twice daily.

In preferred embodiments, the method further comprises a second period, during which giving the subject a drug holiday from administering SEQ ID NO:1. A non-limiting example of the drug holiday comprises withholding SEQ ID NO:1 administration for a period of time in which one or more clinical symptoms of arthritis continue to be alleviated and/or the side effects of treating arthritis continue to be avoided.

In some embodiments, the method further comprises reducing the daily dose of SEQ ID NO:1 during the second period. A non-limiting example comprises reducing the daily dose of SEQ ID NO:1 to from 10 mg to 5 mg per day. In other embodiments, the method further comprises increasing the daily dose of SEQ ID NO:1 during the second period. A non-limiting example comprises increasing the daily dose of SEQ ID NO:1 to from 300 mg to 600 mg per day.

In some embodiments, the second period comprises at least 1 day, at least 2 days, at least 7 days, at least 14 days, at least 42 days, at least 60 days, or until one or more clinical symptoms of arthritis are no longer alleviated in the subject or side effects associated with treating arthritis are no longer avoided.

In preferred embodiments, effectively treating arthritis in a subject comprises alleviating clinical symptoms of arthritis comprising one or more of joint pain, joint stiffness, joint swelling or inflammation, joint tenderness, joint redness, or a limited range of motion. Non-limiting examples comprise one or more of reducing joint pain, reducing joint stiffness, reducing joint swelling, reducing joint redness, or increasing range of motion. In some embodiments, the joint pain, joint stiffness, joint swelling, and/or joint redness are reduced by at least about 20%. In other embodiments, the joint pain, joint stiffness, joint swelling, and/or joint redness are reduced by at least about 50%. In some embodiments, the joint pain, joint stiffness, joint swelling, and/or joint redness are reduced by at least about 80%. In some embodiments, the joint pain, joint stiffness, joint swelling, and/or joint redness are reduced by at least about 100%. In some embodiments, the range of motion is increased by at least about 20%, at least 50%, at least 80%, or at least 100%. Non-limiting examples of clinical measurements for arthritis symptoms may comprise a 0-10 pain scale, a walk or gait test, a grip strength test, a tender joint count, and a swollen joint count.

Non-limiting examples of the side effects associated with treating arthritis comprise gastrointestinal issues, allergic reactions, anemia, headache, edema liver damage, bone marrow suppression, lung infection, osteoporosis, increased risk of blood clots, heart attack, and/or stroke. Gastrointestinal issues may comprise nausea, vomiting, abdominal pain, acid or sour stomach, ulcers, constipation, diarrhea, black, tarry stools, bloody urine, heartburn, and/or belching. Allergic reaction may comprise hives, difficulty breathing, and/or swelling of the face, lips, tongue, and/or throat.

In preferred embodiments, the method of the present invention is for avoiding the one or more side effects commonly associated with treating arthritis. For example, preventing and/or reducing the severity and/or reducing the frequency of one or more gastrointestinal issues, allergic reactions, anemia, headaches, edema, liver damage, bone marrow suppression, lung infection, osteoporosis, risk of blood clots, heart attack, and/or stroke.

In some embodiments, the side effects including gastrointestinal issues, allergic reactions, anemia, headaches, and/or edema are reduced by at least about 20%. In some embodiments, the side effects including gastrointestinal issues, allergic reactions, anemia, headaches, and/or edema are reduced by at least about 50%. In some embodiments, the side effects including gastrointestinal issues, allergic reactions, anemia, headaches, and/or edema are reduced by at least about 80%. In some embodiments, the side effects including gastrointestinal issues, allergic reactions, anemia, headaches, and/or edema are reduced by at least about 100%. Non-limiting examples of avoiding the other side effects associated with treating arthritis may comprise reducing liver damage, bone marrow suppression, lung infection, osteoporosis, risk of blood clots, heart attack, and/or stroke by at least about 20%, at least about 50%, at least about 80%, and at least about 100%.

Non-limiting examples of risk factors in a subject for developing arthritis comprise family history of arthritis or related inflammatory conditions, elevated rheumatoid factor (RA factor), repetitive joint stress, physical joint damage, autoimmune disease, emotional stress, and/or allergens. In other embodiments, the polypeptide comprises a variant sequence of SEQ ID NO:1 in which the variant retains activity about equal to or greater than a polypeptide of SEQ ID NO: 1.

The variant sequence comprises one or more amino acid mutation with respect to SEQ ID NO:1 selected from:

(a) K¹ to A;

(b) P², P⁵, P⁶, or a combination thereof to A;

(c) S³, S⁴, or S³ and S⁴ to A; or

(d) E⁷, E³, or E⁷ and E⁸ to A,

In some embodiments, the method further features the use of an antiproliferative agent is an anti-microtubule agent in combination with SPL-108 or a CD44-modulating peptide. A non-limiting example of an antiproliferative agent is an antimicrotubule agent comprising a taxane. Non-limiting examples of a taxane comprise paclitaxel, docetaxel, cabazitaxel, and albumin-bound paclitaxel. In some embodiments, the paclitaxel is administered intravenously at weekly doses of 80-100 mg/m² on Days 1, 8, and 15 in 28 day cycles, administered intravenously at doses of 135 mg/m² to 175 mg/m² over 3 hours, once every 3 weeks, administered intravenously at doses up to 250 mg/m² over 24 hours, once every 3 weeks, administered intravenously at doses of 120 mg/m² to 140 mg/m² over 96 hours (20 mg/m² per day to 35 mg/m² per day for 5 days continuous IV infusion) once every 3 weeks, or administered intravenously at doses of 80 mg/m² to 100 mg/m² over 1 hour, once a week for each of 3 weeks.

In some embodiments, the method further comprises during a second period, giving the subject a drug holiday from administering the standard-of-care treatment or antiproliferative agent (e.g., withholding agent administration for a period of time). Non-limiting examples of the period of time of withholding the agent administration comprise at least 1 day, at least 2 days, at least 7 days, at least 14 days, or until one or more clinical symptoms reappear in the subject. In some embodiments, the weekly dose of the standard-of-care treatment or antiproliferative agent is reduced during the second period. A non-limiting example comprises reducing the antiproliferative agent to 1 mg/m² to 10 mg/m² per week. In other embodiments, the weekly dose of the standard-of-care treatment or antiproliferative agent is increased during the second period. A non-limiting example comprises increasing the antiproliferative agent to 250 mg/m² to 400 mg/m² per week.

In one aspect is a method of treating arthritis in a patient in need thereof by administering an effective amount of a CD44-modulating polypeptide. In another aspect provided herein is a method of treating arthritis in a patient in need thereof by administering an effective amount of a polypeptide comprising the amino acid sequence Ac-KPSSPPEE-NH2 (SEQ ID NO:1, “A6” or “SPL-108) or NASAPPEE (SEQ ID NO:2) (FIG. 1) in combination with an effective amount of a standard-of-care treatment for arthritis (e.g., NSAIDs, anti-inflammatory agent). In another aspect provided herein is a method of treating arthritis in a patient in need thereof by administering an effective amount of a polypeptide consisting of the amino acid sequence of SEQ ID NO:1 or SEQ ID NO:2 in combination with an effective amount of physical therapy. Amino acids of polypeptides described herein are numbered in reference from their N to C terminal (or its equivalent). For example, SEQ ID NO:1 can be numbered as follows: Ac-K¹P²S³S⁴P⁵P⁶E⁷E⁸-NH2.

The polypeptide of SEQ ID NO:1 can be a capped 8-amino acid peptide. The sequence of the polypeptide of SEQ ID NO:1 corresponds to amino acid residues 136-143 of the connecting peptide domain of human urokinase plasminogen activator (uPA).

The polypeptide of SEQ ID NO:1 shares sequence homology with a portion of the Link-Domain of CD44 (CD44 amino acid residues NASAPPEE occupying positions 120-127; SEQ ID NO:2) (FIG. 1). The CD44 gene is encoded by 20 exons in the mouse and 19 exons in humans. There are 5 constant exons expressed at the 5′ end, and 10 variant exons (mouse) or 9 variant exons (human) can be alternatively spliced within CD44 at an insertion site after the fifth constitutive exon, followed by the remaining constant exons at the 3′ end. The smallest isoform of CD44 (CD44s) contains no variant exons. The largest isoform of CD44 (CD44v1-10) contains all of the variant exons. SEQ ID NO:1 can be found nearly in all CD44 isoforms, in part, because it is located within the first 5 non-variable exons of the isoform. SEQ ID NO:1 is located at the CD44 splice junction of exons 3 and 4.

The SEQ ID NO:1 can include a substitution of K to A; P to A; S to A; or E to A. In some embodiments, the sequence of SEQ ID NO:1 can be modified such that the CD44-modulating polypeptide includes a mutation of K1 to A so long as the polypeptide retains activity about equal to or greater than the polypeptide of SEQ ID NO:1. In another embodiment, SEQ ID NO:1 can be modified to include mutation of P2, P5, P6, or a combination thereof to A so long as the polypeptide retains activity about equal to or greater than the polypeptide of SEQ ID NO:1. In certain embodiments, P2 can be mutated to A. In certain embodiments, P5 can be mutated to A. In certain embodiments, P6 can be mutated to A. In another embodiment, S3, S4, or S3 and S4 can be mutated to A so long as the polypeptide retains activity about equal to or greater than the polypeptide of SEQ ID NO:1. In another embodiment, E7, E8, or E7 and E8 can be mutated to A so long as the polypeptide retains activity about equal to or greater than the polypeptide of SEQ ID NO:1. It is to be understood that the above mutations can be combined in any manner to modify the polypeptide of SEQ ID NO:1 so long as the polypeptide retains activity about equal to or greater than the polypeptide of SEQ ID NO:1. In some embodiments, SEQ ID NO:1 can include at least one glycosylation site. The glycosylation site can be an O-linked glycan on S3, S4, or S3 and S4 of SEQ ID NO:1. In other instances, the glycosylation site can be present in any one Ser or Thr residue of SEQ ID NOs:1-6.

CD44-modulating polypeptides can have anti-migratory activity on joint fibroblast cells (e.g., fibroblast-like synoviocytes; FLSs). In one embodiment, the CD44-modulating polypeptide described herein is a polypeptide comprising or consisting of SEQ ID NO:1 or SEQ ID NO:2 and can have anti-migratory activity on FLSs and leukocytes. A CD44 modulating polypeptide can reduce the migratory activity of a FLS by at least 2, 3, 4, 5, 10, 15, 20, 25, 30, 35, 40, 45, 50, 55, 60, 65, 70, 75, 80, 85, 90, 95, or 100%. For example, the migratory activity of a FLS cell can be reduced by about: 1% to about 95%, 5% to about 95%, 10 to about 95%, 15% to about 95%, 25% to about 95%, 5% to about 100%, 10% to about 100%, 25% to about 100%; 10% to about 80%, or 10% to about 50%. In another example, the migratory activity can be reduced by at least 5, 10, 20, 25, 30, 40, or 50%. Such reductions can be measured against a control sample or, for example, a baseline sample taken from a subject prior to beginning any treatment described herein.

CD44-modulating polypeptides can have anti-invasive activity on FLSs. In one embodiment, the CD44-modulating polypeptide described herein is a polypeptide comprising or consisting of SEQ ID NO:1 or SEQ ID NO:2 and can have anti-invasive activity on FLSs. A CD44 modulating polypeptide can reduce the invasive activity of a fibroblast by at least 2, 3, 4, 5, 10, 15, 20, 25, 30, 35, 40, 45, 50, 55, 60, 65, 70, 75, 80, 85, 90, 95, or 100%. For example, the invasive activity of a fibroblast can be reduced by about: 1% to about 95%, 5% to about 95%, 10 to about 95%, 15% to about 95%, 25% to about 95%, 5% to about 100%, 10% to about 100%, 25% to about 100%; 10% to about 80%, or 10% to about 50%. In another example, the invasive activity can be reduced by at least 5, 10, 20, 25, 30, 40, or 50%. Such reductions can be measured against a control sample or, for example, a baseline sample taken from a subject prior to beginning any treatment described herein.

In some embodiments, treatment methods utilizing SPL-108 polypeptides or the polypeptide of SEQ ID NO:1 or capped variants are useful for inhibiting cell migration and invasion or migration-induced cell proliferation in a subject having a disease or condition associated with undesired cell migration, cell invasion, migration-induced proliferation, angiogenesis or metastasis. Cell migration processes include, for example, the locomotion of a cell from one point to another within the organism. Cell migration is well known in the art and generally involves cytoskeleton polymerization at the leading edge, local detachment from the ECM followed by movement, reattachment and depolymerization. Exemplary cell migration processes occur, for example, during normal development, throughout tissue growth and homeostasis and during aberrant proliferative conditions such as metastasis. For example, tissue formation during embryonic development, wound healing and immune responses all require the movement of cells in a particular direction to a specific location. Similarly, errors during this process can have deleterious consequences, including vascular disease, rheumatoid arthritis, tumor formation, metastasis and mental retardation.

Cell invasion processes include attachment and penetration of a cell into or through a tissue. Cell invasion can be related to cell migration because it involves cell locomotion. However, invasive processes can include a different profile of cell adhesion receptors and/or ECM polypeptides compared to cellular processes that are solely migratory. Cell invasion also can involve other cellular process such as proteolysis and matrix reorganization. An exemplary cell invasion process is the attachment to, proteolysis of penetration of a metastatic cell into or through a blood vessel or the basal lamina. Cellular locomotion into or through, for example, a blood vessel or basal lamina, can occur by cell migration. A variety of other cell invasion processes also are well known in the art.

The table below describes non-limiting examples of polypeptide sequences that may be used in the treatment of arthritis.

| Sequence | SEQ ID NO: |
|---|---|
| Ac-KPSSPPEE-NH₂ | SEQ ID NO: 1 |
| Ac-NASAPPEE-NH₂ | SEQ ID NO: 2 |
| QETWFQNGWQGKNP | SEQ ID NO: 3 |
| KEKWFENEWQGKNP | SEQ ID NO: 4 |
| KEQWFGNRWHEGYR | SEQ ID NO: 5 |
| QIRQQPRDPPTETLELEVSPDPAS | SEQ ID NO: 6 |
| KPSSPPEE | SEQ ID NO: 7 |
| NASAPPEE | SEQ ID NO: 8 |

### EXAMPLE

The following are non-limiting examples of the present invention. It is to be understood that the examples are not intended to limit the present invention in any way. Equivalents or substitutes are within the scope of the present invention.

### Example 1: The Effects of SPL-108 in a Collagen-Induced Arthritis Mouse Model

In the examples below, mice were used in a collagen-induced arthritis (CIA) model, a commonly used model to examine pathogenesis of rheumatoid arthritis. FIG. 3 shows a schematic of CIA induction and development. At day 0, mice were immunized with type II collagen/CFA. At day 18, mice had a booster immunization with type II collagen/IFA. At day 28, the mice had a booster immunization with lipopolysaccharide (LPS; 5 μg). Arthritis onset subsequently occurs 4-10 days after the day 28 LPS injection. The experimental groups consisted of four groups: group 1: 6 CIA mice treated with vehicle; group 2: 6 CIA mice treated with 5 Omg/kg SPL-108 twice a day, starting at arthritis onset (between day 32 to 36); group 3: 6 CIA mice treated with 100 mg/kg SPL-108 twice a day, starting at arthritis onset (between day 32 to 36); and group 4: 6 mice without CIA and not treated (negative control). In preventive strategies, a prophylactic treatment of SPL-108 would be started at day 0, at the time of first immunization. FIG. 4 shows non-limiting examples of a non-diseased paw and a diseased paw of CIA mice, with clinical sores of 0 and 3, respectively.

The pathologic hallmark of rheumatoid arthritis (RA) is a synovial pannus that comprises proliferating and invasive fibroblast-like synoviocytes (FLSs), infiltrating leukocytes, and an associated neoangiogenic response (FIG. 2). Rheumatoid FLSs destroy cartilage and underlying bone by producing matrix metalloproteinases (MMPs), inflammatory and growth-promoting cytokines, and prostaglandins. Rheumatoid synoviocytes also resist apoptosis and show increased adhesive and invasive properties; for instance, when implanted into immunodeficient mice, they readily migrate to distant tissue sites. The tumor-like features of these stromal lineage cells persist during long-term culture and may result from epigenetic and genetic alterations, including mutations in the tumor suppressor p53 that augment pro survival pathways.

### Effects of SPL-108 (A6) on Body Weight in CIA

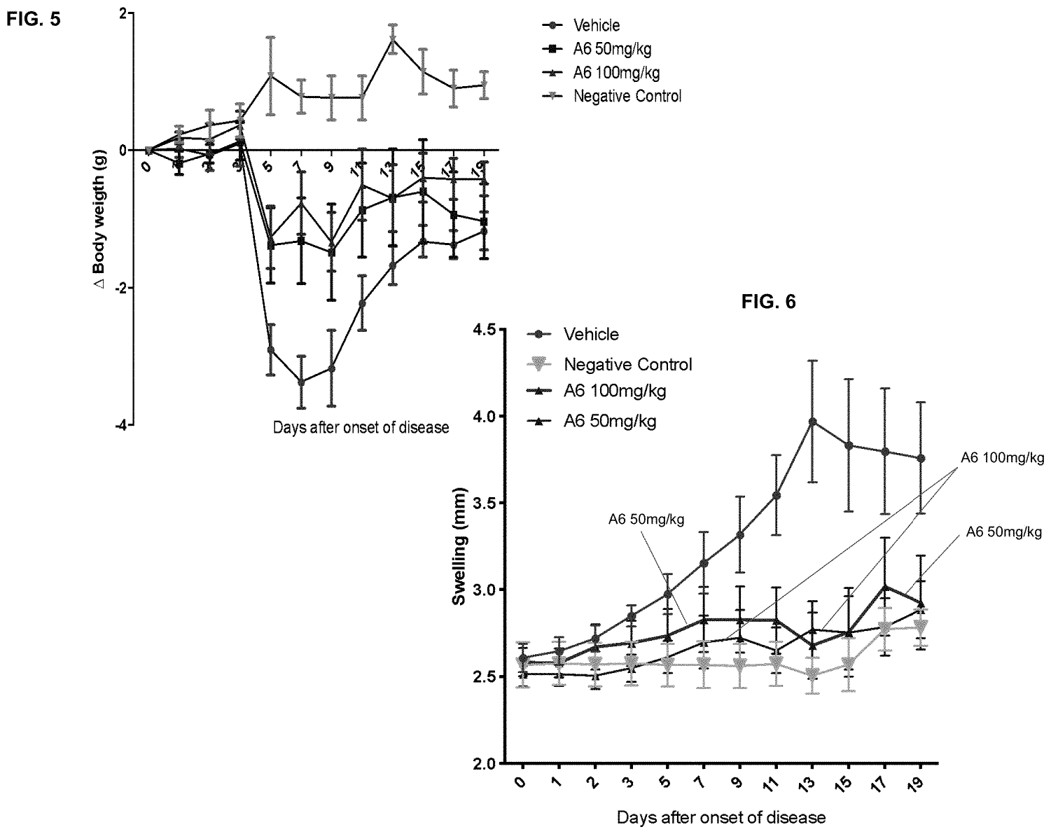

FIG. 5 shows the body weight of mice through 11 days after onset of disease. Although the body weights of all CIA mice started to decrease three days after disease onset, the weight of the mice treated with SPL-108 (A6) did not decrease as much as the weight of the CIA mice treated with only vehicle (˜1 gram vs 3 grams decrease). The weight of the negative control mice did not decrease over the 11 days of measurement. Vehicle vs negative control: significantly different from day 5 to day 19 50 mg/kg vs vehicle: significantly different at day 7 and 9 100 mg/kg vs vehicle: significantly different at day 5, 7, 9 and 11. Thus, SPL-108 alleviated some of the weight loss associated with CIA.

### Effects of SPL-108 (A6) on Joint Swelling in CIA

FIG. 6 shows the swelling of paws (shown as paw size in mm) of mice through 11 days after onset of disease. The paw swelling was lower in the CIA-mice treated with either dose of SPL-108 as compared to administration of vehicle. Vehicle vs negative control: significantly different from day 9 to day 19; 50 mg/kg vs vehicle: significantly different from day 11 to day 19; and 100 mg/kg vs vehicle: significantly different from day 11 to day 19. Thus, SPL-108 alleviated joint swelling associated with CIA.

### Effects of SPL-108 (A6) on Clinical Score in CIA

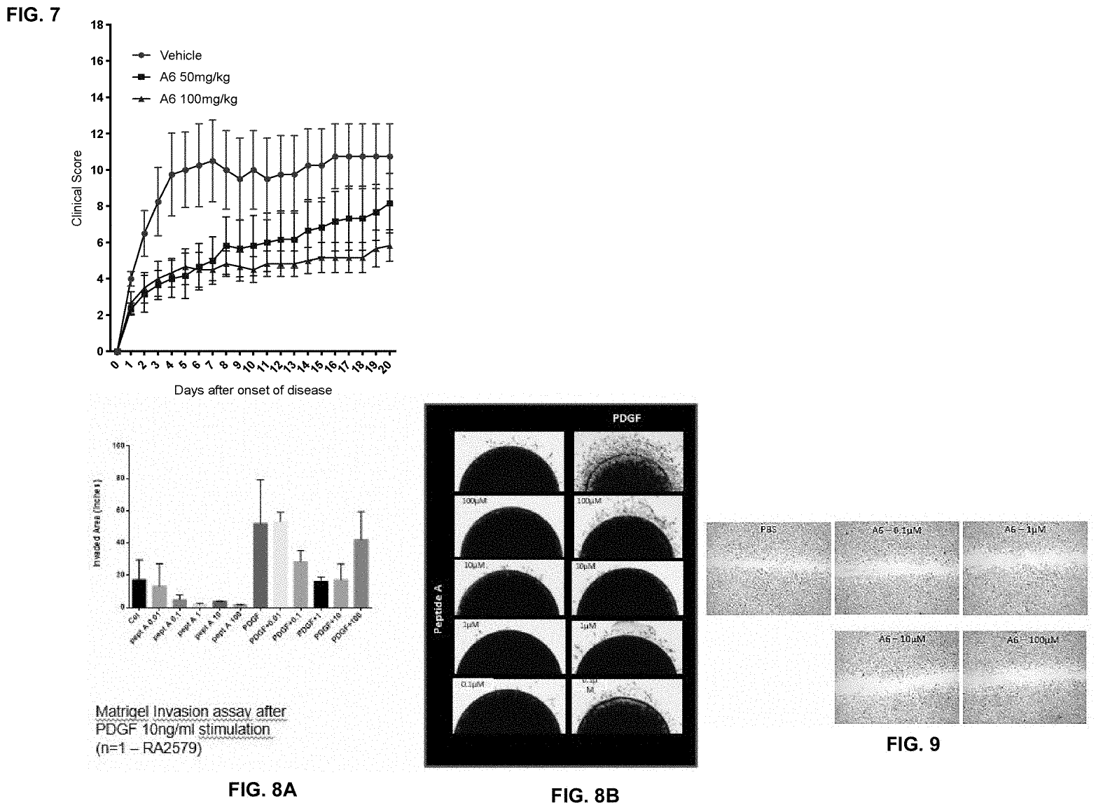

FIG. 7 shows the clinical score of mice through 11 days after onset of disease. The clinical was lower in the CIA-mice treated with either dose of SPL-108 as compared to administration of vehicle. Significance: 50 mg/kg vs vehicle: significantly different at day 4, 5, 6, 7; 100 mg/kg vs vehicle: significantly different from day 4 to day 20. Thus, SPL-108 alleviated joint swelling and tenderness associated with CIA.

### Effects of SPL-108 (A6) on Fibroblast-Like Synoviocytes (FLSs) Invasion

FIG. 8A and FIG. 8B show the effects of SPL-108 (A6) on the invasion of FLSs using an in vitro Matrigel invasion assay. Platelet-derived growth factor (10 ng/mL) was used to stimulate invasion and the area of invasion was measured in inches before and after the addition of 0.1, 1.0, 10.0, and 100 μM SPL-108 (A6) in the presence and absence of PDGF. SPL-108 inhibited the invasion of FLSs in a dose-dependent manner.

### Effects of SPL-108 (A6) on Fibroblast-Like Synoviocytes (FLSs) Migration

FIG. 9 shows the effects of SPL-108 (A6) on the migration of FLSs using an in vitro Scratch migration assay before and after the addition of 0.1, 1.0, 10.0, and 100 μM SPL-108 (A6). Cells were grown to confluence and a thin “wound” was introduced by scratching with a pipette tip. Cells at the wound edge polarize and migrate into the wound space. SPL-108 inhibited the migration of FLSs in a dose-dependent manner.

### Effects of SPL-108 (A6) on Histological Scores in CIA

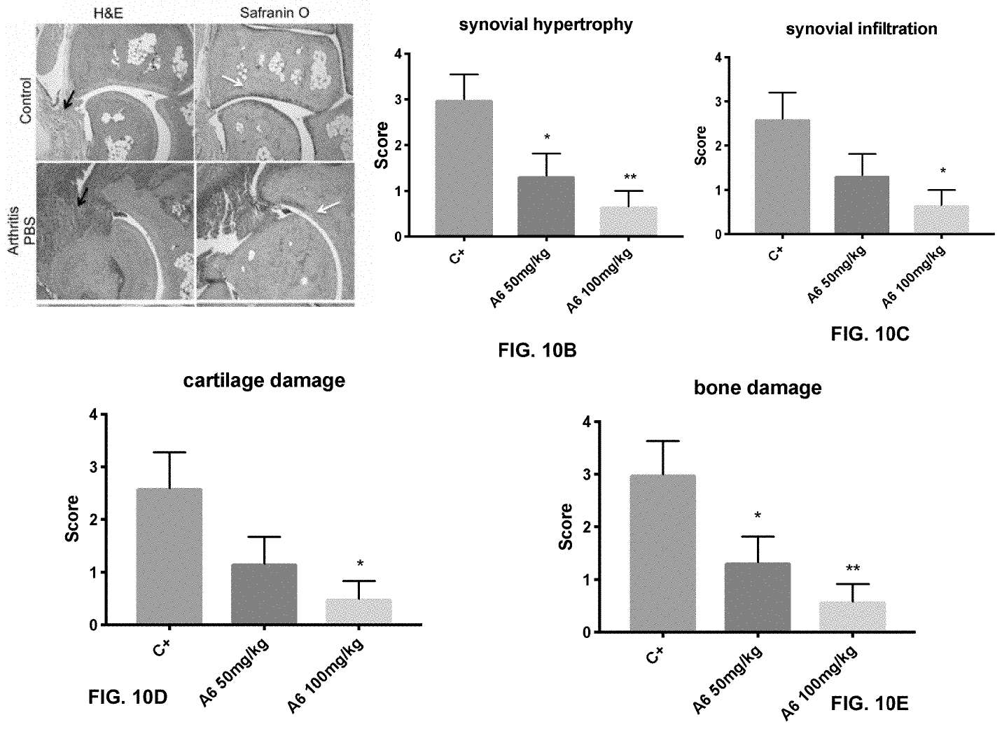

FIGS. 10A, 10B, 10C, 10D, and 10E show histological scores of ankle bones collected at the end of experiments. At the end of the experiment, ankles were collected and decalcified in EDTA for 10 days. They were embedded and sagittal sections were obtained and stained. H&E staining was used to help score cell infiltration, synovial thickness, and bone damage. In the figure, safranin staining will help to score cartilage damage. Black arrows show infiltration in arthritic mice treated with PBS compared to control mice without arthritis. White arrows show lack of safranin staining (red) in the arthritic mice cartilage because of proteoglycan degradation compared to control mice without arthritis. Treatment with SPL-108 resulted in a reduction in synovial hypertrophy, synovial infiltration, cartilage damage and bone damage in a dose dependent manner. Furthermore, this demonstrates that SPL-108 not only affects invasion and migration, but also affects the proinflammatory cytokine response.

### Example 2 Effects of SPL-108 (A6) and Paclitaxel on Osteoarthritis Symptoms in Patients Undergoing Combination Therapy

Two patients diagnosed with endothelial ovarian cancer, and enrolled in a clinical trial for evaluating the efficacy of SPL-108 in combination with paclitaxel (standard-of-care treatment), reported relief from symptoms of pre-existing osteoarthritis. Each patient received SPL-108 150 mg twice a day and Paclitaxel 80 mg/m² weekly on Days 1, 8, and 15, in three 28-day cycles. Each patient received a total of 25,200 mg of SPL-108 over the course of treatment. The improvements in osteoarthritis symptoms included reduction to elimination of joint pain.

Without wishing to limit the present invention to any particular theory or mechanism, it is believed that SPL-108 inhibits both FLS invasion and FLS migration of the immune response in rheumatoid arthritis. The immune response is believed to be initiated/maintained by one or more antigens present in the synovial compartment, which leads to an influx of acute inflammatory cells and lymphocytes into the joint. Subsequent attack chain inflammation lead to the formation of the pannus, which contains FLSs and macrophages that produce proinflammatory cytokines such as TNF-alpha and IL-1. Thus, it is believed that SPL-108 prevents tissue injury by inhibiting the local release of proteolytic enzymes and various inflammatory mediators and activation of osteoclasts. The loss of articular cartilage and formation of bone erosions and subsequent disablement is therefore prevented.

As used herein, the term “about” refers to plus or minus 10% of the referenced number. As used herein, the term “substantially similar to” means plus or minus 20% of the reference number. As used herein “about equal to” refers to plus or minus 5% of the reference number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of treating arthritis or alleviating one or more clinical symptoms of arthritis in a subject in need thereof, said method comprising, during a first period, administering a polypeptide, wherein said polypeptide is Ac-KPSSPPEE-NH₂ (SEQ ID NO:1), an active variant thereof, or a CD44-modulating peptide, to said subject at a daily dose from 10 mg to 300 mg per day; and administering an antiproliferative agent to said subject at a weekly dose of from 10 mg/m² to 250 mg/m² per week, wherein the polypeptide and antiproliferative agent administered during a first period are effective for treating arthritis or alleviating one or more clinical symptoms of arthritis.

2. The method of claim 1, wherein said first period comprises a period of time sufficient to alleviate one or more clinical symptoms of arthritis comprising at least 1 day, at least 2 days, at least 7 days, at least 14 days, at least 42 days, at least 60 days, or until said one or more clinical symptoms of arthritis are no longer alleviated in said subject.

3. The method of claim 1, wherein said arthritis comprises osteoarthritis, reactive arthritis, rheumatoid arthritis, psoriatic arthritis or combination thereof.

4. The method of claim 1, wherein said administration of SEQ ID NO:1 is parenteral administration.

5. The method of claim 1, further comprising, during a second period, giving said subject a drug holiday from administering SEQ ID NO:1.

6. The method of claim 5, wherein said drug holiday comprises withholding SEQ ID NO:1 administration for a period of time.

7. The method of claim 1, further comprising, during a second period, reducing said daily dose of SEQ ID NO:1.

8. The method of claim 1, further comprising, during a second period, increasing said daily dose of SEQ ID NO:1.

9. The method of claim 8, further comprising, during said second period, increasing the daily dose of SEQ ID NO:1 to from 300 mg to 600 mg per day.

10. The method of claim 1, wherein the one or more clinical symptoms of arthritis is selected from: joint pain, joint stiffness, joint swelling, joint redness, reduced range of motion.

11. The method of claim 10, wherein joint pain is reduced by at least about 20%.

12. The method of claim 10, wherein joint stiffness is reduced by at least about 20%.

13. The method of claim 10, wherein joint redness is reduced by at least about 20%.

14. The method of claim 10, wherein range of motion is increased by at least about 20%.

15. The method of claim 1, wherein said polypeptide comprises a variant sequence of SEQ ID NO:1, wherein the variant sequence comprises one or more amino acid mutation with respect to SEQ ID NO:1 selected from:
(a) K¹ to A;
(b) P², P⁵, P⁶, or a combination thereof to A;
(c) S³, S⁴, or S³ and S⁴ to A; or
(d) E⁷, E³, or E⁷ and E⁸ to A,
wherein said mutation retains CD44-modulating activity substantially similar to or greater than said polypeptide of SEQ ID NO:1.

16. The method of claim 1, wherein the antiproliferative agent is an anti-microtubule agent.

17. The method of claim 16, wherein said antimicrotubule agent is a taxane.

18. A method of treating arthritis or alleviating one or more clinical symptoms of arthritis in a subject in need thereof, said method comprising, during a first period, administering a polypeptide, wherein said polypeptide is Ac-KPSSPPEE-NH₂ (SEQ ID NO:1), an active variant thereof, or a CD44-modulating peptide, to said subject at an effective daily dose over a period of time sufficient to effectively treat arthritis or alleviate one or more clinical symptoms of arthritis.

19. The method of claim 18, wherein said polypeptide is Ac-KPSSPPEE-NH₂ (SEQ ID NO:1), and said effective dose is from about 10 mg to about 300 mg per day.

20. The method of claim 18, wherein said polypeptide comprises a variant sequence of SEQ ID NO:1, wherein the variant sequence comprises one or more amino acid mutation with respect to SEQ ID NO:1 selected from:
(a) K¹ to A;
(b) P², P⁵, P⁶, or a combination thereof to A;
(c) S³, S⁴, or S³ and S⁴ to A; or
(d) E⁷, E⁶, or E⁷ and E³ to A,
wherein said mutation retains CD44-modulating activity substantially similar to or greater than said polypeptide of SEQ ID NO:1.


SEQUENCE LISTING (8 sequences)

SEQ ID NO: 1 - protein, 8 aa, Homo sapiens
  MOD_RES (1)..(1): ACETYLATION
  MOD_RES (8)..(8): AMIDATION
KPSSPPEE

SEQ ID NO: 2 - protein, 8 aa, Homo sapiens
  MOD_RES (1)..(1): ACETYLATION
  MOD_RES (8)..(8): AMIDATION
NASAPPEE

SEQ ID NO: 3 - protein, 14 aa, Homo sapiens
QETWFQNGWQGKNP

SEQ ID NO: 4 - protein, 14 aa, Homo sapiens
KEKWFENEWQGKNP

SEQ ID NO: 5 - protein, 14 aa, Homo sapiens
KEQWFGNRWHEGYR

SEQ ID NO: 6 - protein, 24 aa, Homo sapiens
QIRQQPRDPPTETLELEVSPDPAS

SEQ ID NO: 7 - protein, 8 aa, Homo sapiens
KPSSPPEE

SEQ ID NO: 8 - protein, 8 aa, Homo sapiens
NASAPPEE